(19) United States
(12) Patent Application Publication
(10) Pub. No.: US 2008/0119567 A1
(43) Pub. Date: May 22, 2008

(54) Methods of use of herbal compositions

(75) Inventors: Andy A.T. Fong, Taipei (TW); Guang-Tzuu Shane, Taipei (TW); Hsun-Lang Chang, Huallen City (TW); Wei-Ying Kuo, Gueishan Township (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: MEDIGREEN BIOTECHNOLOGY CORP.

(21) Appl. No.: 11/984,617

(22) Filed: Nov. 20, 2007

Related U.S. Application Data
(62) Division of application No. 10/901,693, filed on Jul. 28, 2004.
(60) Provisional application No. 60/491,729, filed on Jul. 29, 2003.

(51) Int. Cl. A61K 31/045; A61P 31/04

(52) U.S. Cl. 514/739

(57) ABSTRACT

The present invention relates to herbal composition and methods of administering an herbal composition, comprising geranium oil and extracts from roots of the plants of the genus Sophora, particularly S. flavescenes and S. tonkinensis, to humans and other mammalian animals with gastric ulcers induced by H. pylori infection. The same composition is shown to be able to eradicate H. pylori in vitro. The invention also relates to a composition comprising citronellol and extracts from roots of Sophora plants. The present invention further relates to the use of citronellol to inhibit gastric ulcers. The invention also relates to the use of the same composition to inhibit the enzymatic activity of Na⁺/K⁺-ATPase to strengthen heart muscle contractions and prevent heart failure.

This application claims the benefit of U.S. Provisional Application No. 60/491,729, filed Jul. 29, 2003, which is incorporated herein by reference.

### BACKGROUND OF THE INVENTION

1. Field of Invention

This invention relates generally to the use of herbal compositions in people with gastric ulcers, and more particularly, in Helicobacter pylori infection related gastric ulcers. This invention also relates generally to the use of herbal compositions in people with heart failure, and more particularly, to the inhibition of the enzymatic activity of Na⁺/K⁺-ATPase.

2. Description of Related Art

Peptic ulcers are erosions of mucous membranes in the lower part of the esophagus, the stomach, the duodenum, and the jejunum. The most common forms of peptic ulcers are duodenal and gastric ulcers, accounting for 80% and 16% of all peptic ulcers, respectively. There are three major causes of peptic ulcers: infection, certain types of medication, and disorders that cause over secretion of stomach juices. Infection with H. pylori has been found to be the cause of 90% of duodenal ulcers and 80% of gastric ulcers.

H. pylori is a spiral shaped gram-negative bacterium that lives in the mucous tissues that line the digestive tract. The majority of peptic ulcer patients are given H2 blockers and proton pump inhibitors to reduce stomach acid secretion in order to relieve the ulcers symptoms and heal gastric mucosal inflammation. However, the bacterial infection is not treated. Currently, the medical community is turning to eradication of the bacteria as part of the treatment plan for peptic ulcers. Therapy for H. pylori infection consists of one to two weeks of one or two antibiotics, such as amoxicillin, tetracycline, metronidazole, or clarithromycin, plus either ranitidine bismuth citrate, bismuth subsalicylate, or a proton pump inhibitor to reduce stomach acid secretion. Such a treatment plan relies heavily on the use of antibiotics and involves the administration of a combination of drugs. The use of antibiotics may not be successful with some patients due to antibiotic resistance.

Heart failure, a condition in which the heart is unable to pump blood at an adequate rate or in adequate volume, has been treated by drugs that inhibit Na⁺/K⁺-ATPase, i.e. quabain, gitaligin, digihermin, digoxin, digicoside, digitoxin, digitamin, and lanatoside C. The inhibition of Na⁺/K⁺-ATPase can lead to an increase in the concentration of Na⁺ and Ca⁺ inside the heart muscle cells and thus strengthens the contraction of heart muscles. Na⁺/K⁺-ATPase is an enzyme involved in the hydrolysis of ATP to provide the energy necessary for Na⁺/K⁺ pumps, which are found in the plasma membrane of nearly all eukaryotic cells and are especially abundant in kidney and brain tissues and cardiac ventricular muscle cells. By inhibiting the enzymatic activity of Na⁺/K⁺-ATPase, the Na⁺/Ka⁺ pump is then unable to pump out the Na⁺ ions, leading to an increase of Ca⁺ ion concentration inside the cell.

Drugs that inhibit Na⁺/K⁺-ATPase can lead to various side effects such as nausea, headache, visual impairment, mental confusion etc.

### BRIEF SUMMARY OF THE INVENTION

The present invention provides a composition to be used in a method of eradicating H. pylori that causes gastric ulcers. The present invention also provides another composition to be used in a method of inhibiting the enzymatic activities of Na⁺/K⁺-ATPase. The present invention further provides for methods of administering those compositions.

To achieve the objective of the present invention as embodied and broadly described herein, one embodiment of the present invention is drawn to a method of treating a mammal infected with H. pylori comprising administering to the mammal a composition comprising geranium oil and extracts from the root of Sophora plants.

The method of the present invention further provides the route of oral administration, intraperitoneal administration, and intravenous administration. The present invention further provides for a method that uses the composition in the form of oil capsules, tablet, pills, pastes, liquid, syrup, decoction soup, powders, edible form of the Pelargonium and Sophora plants taking together or separately, injections, health food, food additives, or dietary supplement.

The present invention further employs a composition wherein the geranium oil is extracted from P. graveolens, P. roseum, P. terebinthinceum, or one or more species of the genus Pelargonium. The method of the present invention further comprises employing a composition comprising geranium oil, matrine, and oxymatrine. In another embodiment of the present invention, the method employs the composition comprising citronellol, geraniol, citronellyl formate, geranyl formate, matrine, and oxymatrine.

In another embodiment of the present invention, the composition comprises citronellol, geraniol, citronellyl formate, geranyl formate, and extracts from root of at least one plant selected from a group comprising Sophora flavescenes, Sophora tonkinensis, Sophora subprostrata, Sophora alopecuroides, Sophora moorcroftiana, and Euchresta strigillosa. In a further embodiment of the present invention, the composition comprises geranium oil and extracts from the root of at least one plant selected from a group comprising Sophora flavescenes, Sophora tonkinensis, Sophora subprostrata, Sophora alopecuroides, Sophora moorcroftiana, and Euchresta strigillosa.

The present invention also employs a method wherein the composition comprises geranium oil and extracts from the root of S. tonkinensis. In another embodiment of the invention, the composition comprises citronellol, geraniol, geranyl formate, citronellyl formate, linalool, trans-rose oxide, cis-rose oxide, matrine, oxymatrine, and sophocarpine.

The method of the present invention employs a composition, wherein the composition comprises A and B, wherein A is selected from a group consisting of hexanol, 3-hexen-1-ol, α-pinene, β-pinene, P-cymene, limonene, 1,8-cineol, ocimene, linallol oxide, linallol, trans-rose oxide, cis-rose oxide, citronellal, menthone, iso-methone, menthol, terpineol, citronellol, geraniol, citronellyl formate, geranyl formate, caryophellene, citronellyl propinoate, gurjunene, cadiene, and B is selected from a group consisting of matrine, oxymatrine, anagyrine, methylcytisine, cytosine, sophocarpine, sophocarpine N-oxide, sophoramine, sophoranol, sophoranone, sophoradin, sophoranochromene, sophoradochromene, pterocarpine, genistein, maackian, trifolirhizin, sitosterol, lu-peol, and alkyl alcohol ester.

In a particular embodiment of the present invention, the composition comprises geranium oil and extracts from the root of Sophora flavescenes. In yet another embodiment of the present invention, the composition comprises citronellol, geraniol, geranyl formate, citronellyl formate, linalool, trans-rose oxide, cis-rose oxide, kurarinol, matrine, oxymatrine, and sophocarpine. The method according to the present invention further employs a composition that comprises A and B, wherein A is selected from a group consisting of hexanol, 3-hexen-1-ol, α-pinene, β-pinene, P-cymene, limonene, 1,8-cineol, ocimene, linallol oxide, linallol, trans-rose oxide, cis-rose oxide, citronellal, menthone, iso-methone, menthol, terpineol, citronellol, geraniol, citronellyl formate, geranyl formate, caryophellene, citronellyl propinoate, gurjunene, cadiene, and B is selected from a group consisting of matrine, oxymatrine, sophoranol, N-methylcytisine, anagyrine, baptifoline, sophocarpine, sophoridine, iso matrine, 7,11-dehydromatrine, sophoramine, 7-dehydrosophoramine, 9α-hydroxy-sophoramine, 5α,9α-dihydroxymatrine, N-oxysophocarpine, sophoranol N-oxide, rhombifoline, lupanine, mamanine, kuraramine, isokuraramine, and kurarinol.

The method of the present invention employs the composition wherein the effective human dosage is in a range between about 285 mg/60 kg/day and about 4,675 mg/60 kg/day. In another embodiment of the present invention, the effective dosage has a ratio with geranium oil in the range of between about 97% and about 99% and extracts from roots of Sophora flavescenes in the range of between about 3% and about 0.6%.

In another embodiment of the present invention, a composition comprising geranium oil, extracts from the root of S. flavescenes, and excipients is used to treat a mammal infected with H. pylori. In a further embodiment, the composition is in a dosage in a range of between about 300 mg/kg/day to about 600 mg/kg/day.

The present invention provides for a method of inhibiting H. pylori growth comprising delivering to H. pylori a composition comprising geranium oil and extracts from the root of Sophora plants. The invention further provides delivering the composition to H. pylori growth in a human. Another embodiment further provides using a composition with geranium oil extracted from one or more species of the genus Pelargonium, geranium oil extracted from a plant of the genus Pelargonium and species graveolens, geranium oil extracted from a plant of the genus Pelargonium and species roseum, and geranium oil extracted from a plant of the genus Pelargonium and species terebinthinceum.

In another embodiment, the method uses a composition comprising geranium oil, matrine, and oxymatrine. The method of the present invention further employs a composition comprising citronellol, geraniol, citronellyl formate, geranyl formate, matrine, and oxymatrine. The method of the present invention further employs a composition comprising citronellol, geraniol, citronellyl formate, geranyl formate, and extracts from root of at least one plant selected from a group comprising Sophora flavescenes, Sophora tonkinensis, Sophora subprostrata, Sophora alopecuroides, Sophora moorcroftiana, and Euchresta strigillosa.

Alternatively, the method uses a composition comprising geranium oil and extracts from the root of at least one plant selected from a group comprising Sophora flavescenes, Sophora tonkinensis, Sophora subprostrata, Sophora alopecuroides, Sophora moorcroftiana, and Euchresta strigillosa. The method of the present invention further uses a composition comprising geranium oil and extracts from the root of Sophora tonkinensis. In another embodiment of the present invention, the composition comprises citronellol, geraniol, geranyl formate, citronellyl formate, linalool, trans-rose oxide, cis-rose oxide, matrine, oxymatrine, and sophocarpine. In the method of the present invention, the composition comprises A and B wherein A is selected from a group consisting of hexanol, 3-hexen-1-ol, α-pinene, β-pinene, P-cymene, limonene, 1,8-cineol, ocimene, linallol oxide, linallol, trans-rose oxide, cis-rose oxide, citronellal, menthone, iso-methone, menthol, terpineol, citronellol, geraniol, citronellyl formate, geranyl formate, caryophellene, citronellyl propinoate, gurjunene, cadiene, and B is selected from a group consisting of matrine, oxymatrine, anagyrine, methylcytisine, cytosine, sophocarpine, sophocarpine N-oxide, sophoramine, sophoranol, sophoranone, sophoradin, sophoranochromene, sophoradochromene, pterocarpine, genistein, maackian, trifolirhizin, sitosterol, lu-peol, and alkyl alcohol ester.

In another embodiment of the present invention, the composition comprises geranium oil and extracts from the root of S. tonkinensis having a weight ratio of about 30:1. The method of the present invention further comprises a composition comprising about 10% S. tonkinensis powders and about 90% geranium oil powders. The method of the present invention employs a composition comprising about 30% S. tonkinensis powders and about 70% geranium oil powders. In yet another embodiment of the present invention, the concentration is at least about 300 μg/ml, including the weight of the excipients. In another embodiment of the present invention, the concentration is between about 300 μg/ml to about 30 mg/ml, including the weight of the excipients.

The method of the present invention employs a composition comprising geranium oil and extracts from the root of Sophora flavescenes. The composition used in the method of the present invention further comprises citronellol, geraniol, geranyl formate, citronellyl formate, linalool, trans-rose oxide, cis-rose oxide, kurarinol, matrine, oxymatrine, and sophocarpine. The method of the present invention employs a composition, wherein the composition comprises A and B, wherein A is selected from a group consisting of hexanol, 3-hexen-1-ol, α-pinene, β-pinene, P-cymene, limonene, 1,8-cineol, ocimene, linallol oxide, linallol, trans-rose oxide, cis-rose oxide, citronellal, menthone, iso-methone, menthol, terpineol, citronellol, geraniol, citronellyl formate, geranyl formate, caryophellene, citronellyl propinoate, gurjunene, cadiene, and B is selected from a group consisting of matrine, oxymatrine, sophoranol, N-methylcytisine, anagyrine, baptifoline, sophocarpine, sophoridine, iso matrine, 7,11-dehydromatrine, sophoramine, 7-dehydrosophoramine, 9α-hydroxy-sophoramine, 5α,9α-dihydroxymatrine, N-oxysophocarpine, sophoranol N-oxide, rhombifoline, lupanine, mamanine, kuraramine, isokuraramine, and kurarinol.

The present invention also provides a method of treating a mammal infected with H. pylori comprising administering to the mammal a composition comprising citronellol. More specifically, the mammal is a human. In one specific embodiment, the composition is administered orally. In another embodiment, the dosage used is about 25 mg/kg, and more specifically, the composition is administered twice a day. In another embodiment, the dosage for human is 150 mg/60 kg (calculation based on: 25 mg/kg times 60 and divided by 10, which human dosage conversion is well known in the art).

The invention provides a method of inhibiting H. pylori growth comprising delivering a composition to H. pylori comprising citronellol.

The invention also provides for a method of preventing gastric ulcers induced by H. pylori comprising administering to a mammal a composition comprising geranium oil and extracts from the root of Sophora plants.

The invention further provides a method for administering a composition comprising the steps of identifying a mammal suffering from heart failure, determining a route of administering the composition to the mammal, determining a form of the composition to be administered to the mammal, determining a dosage of the composition wherein the composition comprises geranium oil and extracts from the root of S. flavescenes, and delivering the dosage of the composition to the mammal suffering from heart failure.

In another embodiment of the present invention, a method for administering a composition comprises the steps of identifying a mammal suffering from heart failure, determining a route of administering the composition to the mammal, determining a form of the composition to be administered to the mammal, determining a dosage of the composition wherein the composition comprises geranium oil and extracts from the root of S. tonkinensis, and delivering the dosage of the composition to the mammal suffering from heart failure.

Another embodiment of the present invention provides a method for inhibiting Na⁺/K⁺-ATPase comprising contacting Na⁺/K⁺-ATPase with a composition comprising geranium oil and extracts from the root of Sophora plants.

### BRIEF DESCRIPTION OF DRAWINGS

FIG. 1 shows the compounds identified and their relative contents in the geranium oil produced in Kunming, China by the methods of gas chromatography/mass spectroscopy.

FIG. 2 shows the result of pharmcokinetics study of intravenous injection of matrine alone and matrine with and addition of geranium oil in mice.

FIG. 3 shows the result of pharmcokinetics study of intravenous injection of oxymatrine alone and oxymatrine with the addition of geranium oil in mice.

FIG. 4 shows a reduction in the amount of geranium oil used when extracts from S. flavescenes is added.

FIG. 5 shows the result of an LD₅₀ experiment involving oral administration of a composition capsule (300-400 grams of roots of S. flavescenes roots plus 350-450 grams of geranium oil) of the present invention in capsule form to mice.

FIG. 6 shows the calculation of dosage of geranium oil plus extracts from S. flavescenes only, without the excipients.

### DETAILED DESCRIPTION OF THE INVENTION

The present invention relates to methods of using an herbal composition made from geranium oil and extracts from the root of Sophora plants, preferably S. flavescenes or S. tonkinensis, to treat gastric ulcers related to the infection of H. pylori. In addition, the invention relates to the use of citronellol to treat gastric ulcers. Citronellol can be found in many plants and is a major constituent of geranium oil. It can also be synthetically synthesized.

The present invention also relates to methods of using the herbal composition made from geranium oil and extracts from the root of Sophora plants to inhibit Na⁺/K⁺-ATPase in the treatment of heart failure.

1. Geranium Oil

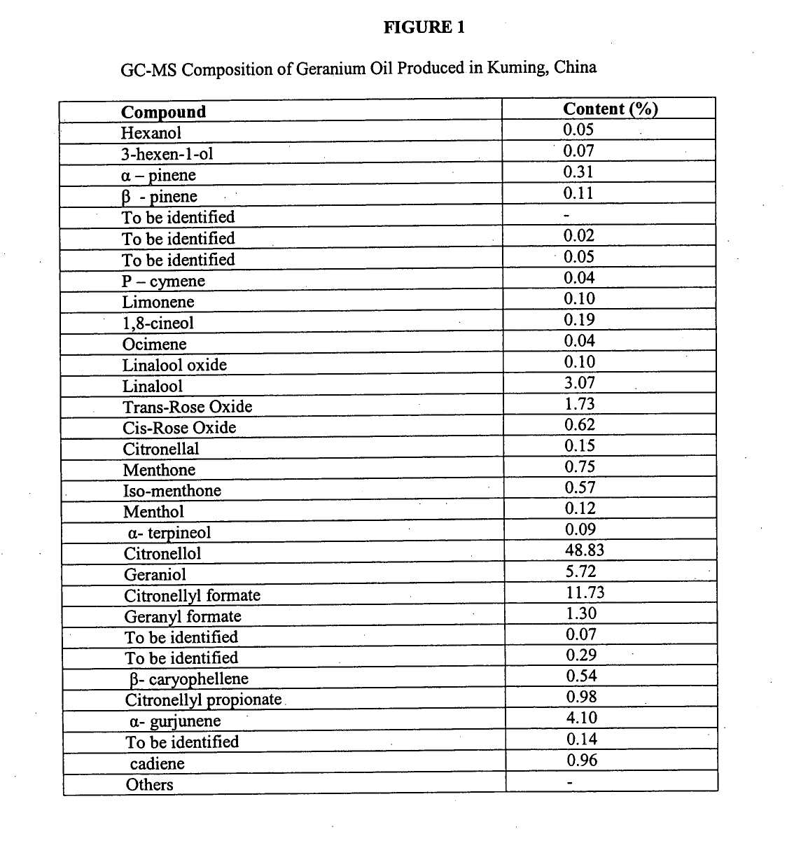

Geranium oil may be collected from steam distillation of the stem and leaves of the plant of division Magnoliophyta, class Magnoliopsida, order Geraniales, family Geraniaceae, and genus Pelargonium. Pelargoniums are native to South Africa and there are more than one hundred species in existence today, including hybridized garden species. Pelargoniums are now grown, and geranium oil is now produced, mainly in Algeria, Egypt, Morocco, Bourbon, China, and Australia. The present invention preferably uses geranium oil extracted from Pelargonium graveolens or Pelargonium roseum and Pelargonium terebinthinceum grown in Kunming City of the Yunan Province in China. FIG. 1 shows a gas chromatography/mass spectroscopy (GC-MS) analysis of the geranium oil produced in Kunming shows the constituent compounds and their relative content, including citronellol, see FIG. 1. The generally known main constituents of geranium oil are citronellol, geraniol, geranyl formate, citronellyl formate, linalool, trans-rose oxide, and cis-rose oxide.

Certain specifications of geranium oil are set out in the National Standard of the People's Republic of China—GB 11959-89, which is incorporated herein by reference, including any drawings. The specifications adopts the same international standard of ISO 4731:1978 Oil of Geranium (Geranium Oil Standard) incorporated by reference. The Geranium Oil Standard specifies the outward characteristics of geranium oil, i.e. the geranium oil takes on a clear oil liquid form of a yellow, greenish, or amber color and has a distinct aroma. The same standard also specifies a relative density of 0.881-0.900 g/cm³, an optical rotation of −6° to −14°, and a refractive index of 1.459-1.466 for geranium oil. In addition, a method, using acetylation and saponification, is prescribed by the same Geranium Oil Standard to determine the total alcohol content of geranium oil. The total alcohol content, determined in accordance with the method prescribed by the Geranium Oil Standard, should be at least 65% (65% alcohol content is calculated as geraniol).

2. Sophora Root

a. S. flavescenes

S. flavescenes typically is about 10-30 cm long, 1-2 cm in diameter, and generally takes on a grayish brown or grayish yellow color. The root preferably has a mild scent and an extremely bitter taste. It is grown mainly in China, Korea, and Japan. Presently, the alkaloids identified in the roots of S. flavescenes are matrine, oxymatrine, sophoranol, N-methylcytisine, anagyrine, baptifoline, sophocarpine, sophoridine, iso matrine, 7,11-dehydromatrine, sophoramine, 7-dehydrosophoramine, 9α-hydroxy-sophoramine, 5α,9α-dihydroxymatrine, N-oxysophocarpine, sophoranol N-oxide, rhombifoline, lupanine, mamanine, kuraramine, isokuraramine, kurarinol. The known main constituents are matrine, oxymatrine. The principal main constituents of S. flavescenes are also found in Sophora subprostrata, Sophora tonkinensis, Sophora alopecuroides, Sophora moorcroftiana, and Euchresta strigillosa.

To ensure the quality of the S. flavescenes roots used, the roots preferably are first checked for their outer appearance. Thin layer chromatography testing is preferably also applied in accordance with the S. flavescenes root identification method as promulgated in the Pharmacopoeia of the People's Republic of China, Appendix VI B (incorporated herein by reference in its entirety, including any drawings) to determine presence of matrine, oxymatrine and sophocarpine. A titration method as prescribed by the Pharmacopoeia of the People's Republic of China for the determination of the total alkaloid content of roots of S. flavescenes may be applied. The total alkaloid content preferably should not be less than 2%. S. flavescenes roots used in the present invention preferably have a total alkaloid content of about 2.74% to 3.03%.

b. S. tonkinensis

The root of S. tonkinensis takes on a long curved tubular form with branches and is typically about 0.3-1.5 centimeters in diameter. The root is hardened and difficult to break. Its surface color ranges from grayish brown to suntan brown with longitudinal wrinkles and holes. The root has a bean scent and is extremely bitter. It is grown mainly in parts of China, i.e. the Guangdong province, Guangxi province, Guizhou province, Yunan province, and Jiangxi province.

The root contains 0.93% of alkaloids, of which 0.52% is matrine and 0.35% is oxymatrine. The other alkaloids identified in the root of Sophora tonkinensis are anagyrine, methylcytisine, cytosine, sophocarpine, sophocarpine N-oxide, sophoramine, and sophoranol. The flavonic compounds identified in the root are sophoranone, sophoradin, sophoranochromene, sophoradochromene, pterocarpine, genistein, maackian, trifolirhizin, sitosterol, lu-peol, and a group of alkyl alcohol ester.

The principal alkaloid constituents of Sophora tonkinensis are also found in Sophora flavescenes, Sophora alopecuroides, Sophora moorcroftiana, and Euchresta strigillosa.

3. Compositions

The use of geranium oil together with the root of Sophora plants can achieve better effect than using either one alone.

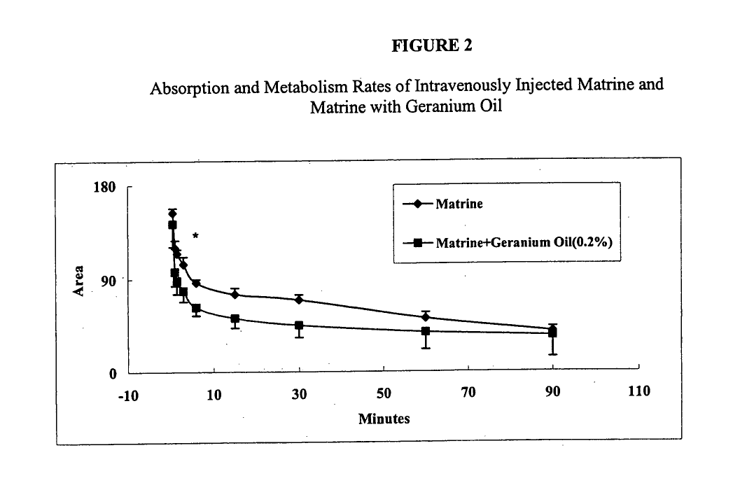

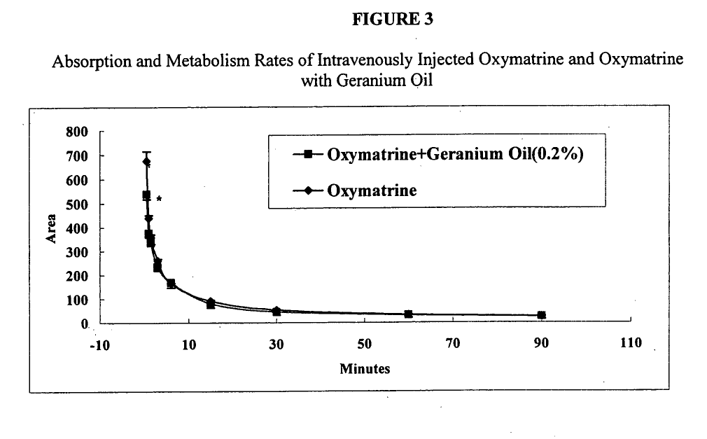

Result of pharmcokinetics study shows that in intravenous injections, the addition of geranium oil to matrine or oxymatrine, the principal constituents of the extracts from the roots of Sophora plants, will increase the absorption and metabolism of the respective compound (FIG. 2 and FIG. 3 show the changes in HPLC peak areas of matrine and matrine plus geranium oil over time in mice).

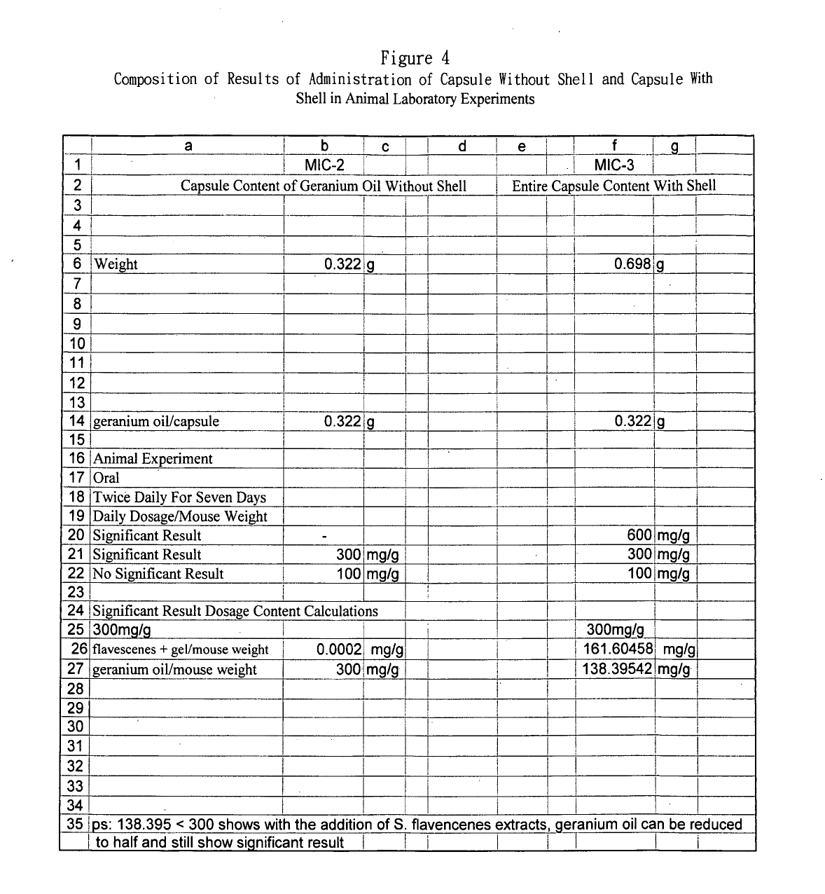

FIG. 4 shows the consumption of the extracts of S. flavescenes roots together with geranium oil will achieve the same result of inducing protectant activity against gastric lesions induced by H. pylori as using geranium oil alone. The addition of the extracts of S. flavescenes can reduce the use of geranium oil to less than half of the original amount of the geranium oil used to achieve the same result. As the price of geranium oil is more than double the price of the root of S. flavescenes, one practical advantage of reducing the amount of geranium oil used is to reduce the cost of the botanical product while achieving the same result. In addition, the reduction of geranium oil makes the botanical product even safer as the geranium oil dosage used would be much less than the maximum tolerable dosage.

Citronellol alone also has the effect of inhibiting gastric lesions induced by H. pylori. The use of a single compound for treatment greatly reduces the cost and effort than that involved with a composition. Citronellol, a major constituent of geranium oil, is easier and cheaper to obtain than geranium oil itself. Citronellol is also present in many other plants and can be synthetically synthesized. It has less regulatory concerns as it can be used as a food additive under food and drug regulations.

a. Capsules

After examining the geranium oil and the S. flavescenes roots for compliance with the specifications as described above, the composition can be made into an oil capsule through the following preferred steps. About 1,000 capsules can be made from the amount of the ingredients described below. 300 to 400 grams of S. flavescenes roots is mixed thoroughly with ethanol in an amount of 1/10 of the weight of the S. flavescenes roots, and then the mixture is smothered for about 12-15 hours. Then the S. flavescenes roots are dried on low heat. The dried S. flavescenes roots are then ground into powder and filtered through 40 mesh. The filtered through S. flavescenes roots powder is then added to 70%-80% ethanol, in an amount of 10 times the weight of the filtered S. flavescenes roots powder. The mixture is in a steam distillation bottle and heated and refluxed for 2 to 4 hours. The solution is removed by filtration and placed aside. Ethanol, in an amount of 6 times the weight of the filtered S. flavescenes root powder, is added to the steam distillation bottle with the S. flavescenes root powder and heated and refluxed for the second time for another 2-4 hours. The solution is filtered, and the two filtered liquids are combined and added to the ethanol collector to condense and collect ethanol and to obtain the S. flavescenes paste (which is of a brownish yellow color and tastes extremely bitter).

The S. flavescenes paste preferably should be tested for its total alkaloid content using the S. flavescenes roots extracts content determination method specified in the Pharmaceutical Product Standard of Heilongiang Province (incorporated herein by reference in its entirety, including any drawings). The total alkaloid content is about 70% to 73% (calculated as oxymatrine). The paste then is dissolved with distilled water, and then 5 to 7 grams of glycerine and 250 to 270 grams of gelatin are added (mixture). After the mixture of S. flavescenes paste, glycerine, and gelatin is completely dissolved, it is placed in the vacuum melting bottle to eliminate the air bubble and the water content until the viscosity reaches about 30-50 pa·s. The mixture of S. flavescenes paste, glycerine, and gelatin and 350 to 450 grams of geranium oil are separately inserted into a capsule making machine. Wherein the mixture of S. flavescenes paste, glycerine, and gelatin forms the capsule shell with geranium oil filling the inside of the composition capsule. The capsules are then parched at 35° C. to 45° C. for 10-15 hours. The total alkaloid content of the entire capsule is about 2% to 10% total alkaloid/capsule, as determined by an analysis of the capsule shell by the spectrophotometric method of the Pharmacopoeia of the People's Republic of China, Appendix VA, incorporated herein by reference.

The S. flavescenes paste may be mixed with glycerol soylecithin and then mixed with geranium oil to produce a form of emulsion for oral intake, or alternatively, a paste form of the composition may be made. Cyclodextrin may also be used to make tablets or pills enclosing the composition. The composition can also be made into dietary supplement, health food (functional food), and food additives. One can also decoct the Pelargonium plant and S. flavescenes roots to obtain a liquid form of the composition for direct oral intake as a medicinal soup or for making into syrup or other forms of liquid composition. S. flavescenes roots and the Pelargonium plant can also be taken orally, in an edible form, separately at a timed interval.

b. Injections

The composition can also be prepared for injections through the following preferred steps. S. flavescenes roots and geranium oil should be examined for compliance with the specifications as stated above. The S. flavescenes roots are ground into coarse powder. Three hundred (300) grams of the S. flavescenes roots powder is added to 1200 ml of geranium oil in a 2000 ml glass heating tube to heat and reflux at 115° C. for 6 hours, and then the liquid is filtered to obtain 800 ml of dark yellow clear liquid oil. The oil liquid is placed in a pestle bowl and Tween 80® in 5% dextrose is slowly added to the bowl while grinding at the same time until the oil liquid becomes transparent and its pH is 6.8 to 7.0. The solution is then filtered, and the filtered solution is placed in a 2 ml ampoule. The ampoule is then sealed and sterilized at 110° C. The compositions can be delivered through intravenous or intraperitoneal injections.

c. Powders

The composition can be formed into powders (powder composition) through the following steps. First, geranium oil and the root of S. tonkinensis or S. flavescenes are prepared separately. β-cyclodextrin is added to geranium oil to prevent evaporation, and excipients are added subsequently to form geranium oil powders. The geranium oil and the excipients are about 31% and 69% by weight, respectively, of the geranium oil powders. Next, the root of S. tonkinensis or S. flavescenes is cut into thin pieces and then ground. About 250 grams of the ground S. tonkinensis or S. flavescenes root is mixed with 3000 ml of water, about 12 times the weight of the ground root. The mixture is then boiled in a steam distillation bottle to heat and reflux for about 1 hour. Afterwards, the scum on the surface of the liquid is removed, and the liquid is filtered through a 100 mesh screen. The filtered liquid is then concentrated and about 66 grams of solid extracts of S. tonkinensis or S. flavescenes is obtained.

Excipients are added to the solid extracts to form S. tonkinensis or S. flavescenes root powders. The S. tonkinensis or S. flavescenes extracts and the excipients are about 60% and 40% by weight, respectively, of the total powders. Subsequently, the geranium oil powders and the S. tonkinensis or S. flavescenes root powders are mixed together with additional excipients to form the composition of the present invention into powder forms, i.e. the powder composition, wherein the geranium oil powders, S. tonkinensis or S. flavescenes root powders, and the excipients are about 56%, 1%, and 43% by weight, respectively, of the powder composition. The weight ratio of geranium oil and extracts of S. tonkinensis or S. flavescenes within the composition are about 30:1. The excipients used in the process to form powders can be starch, sugar, fructose, sorbital etc. and other pharmaceutical excipients commonly used by one skilled in the art. Alternatively, the geranium oil powders and the S. tonkinensis powders are simply mixed together to form a mixture of powders wherein the S. tonkinensis powders and the geranium oil powders are about 10% and 90% respectively or 30% and 70% respectively.

In the alternative, the geranium oil powders and the S. tonkinensis or S. flavescenes root powders can be mixed with glycerine and gelatin to form capsules. The composition can also be made into dietary supplement, health food (functional food), and food additives. One can also decoct the Pelargonium plant and S. tonkinensis or S. flavescenes roots to obtain a liquid form of the composition for direct oral intake as a medicine soup or for making into syrup or other forms of liquid composition. S. tonkinensis or S. flavescenes plant roots and the Pelargonium plant can also be taken orally, in an edible form, separately at a timed interval.

The term, excipients, as used herein broadly refers to pharmaceutically inert substance employed in formation of compositions for intake in any manner.

4. Single Compound

The citronellol compound used is purchased from SunTen Phytotech Co., Ltd. (Taipei, Taiwan).

5. Gastric Ulcer Prevention and Na⁺/K⁺-ATPase Inhibition

Geranium oil and the root of Sophora plants can be used in combination to treat gastric ulcers induced by H. pylori. In the alternative, a single compound of citronellol can be used to inhibit gastric ulceration.

The composition of geranium oil and the root of Sophora plants is also found to have the ability to inhibit the enzymatic activity of Na⁺/K⁺-APTase, leading to increased Ca⁺ inside the cardiac cell and thus strengthening heart contraction.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

Further, all numbers expressing quantities of ingredients, reaction conditions, % purity, and so forth, used in the specification and claims, are modified by the term “about,” unless otherwise indicated. Accordingly, the numerical parameters set forth in the specification and claims are approximations that may vary depending upon the desired properties of the present invention. Nonetheless, the numerical values set forth in the specific examples are reported as precisely as possible. Any numerical value, however, inherently contains certain errors from the standard deviation of its experimental measurement.

It must be noted that, as used herein and in the appended claims, the singular forms “a,” “or,” and “the” include plural referents unless the context clearly dictates otherwise.

The following examples further illustrate the invention. They are merely illustrative of the invention and disclose various beneficial properties of certain embodiments of the invention. The following examples should not be construed as limiting the invention.

### EXAMPLES

The practice of the present invention will employ, unless otherwise indicated, conventional techniques of biology and Chinese medicine, which are within the skill of the art. Such techniques are explained fully in the literature.

The following examples illustrate the gastric ulcer inhibition function of the composition of geranium oil and the extracts of root of Sophora plants and of the citronellol compound alone. The following examples also illustrate the APTase inhibition effect of the composition of geranium oil and the extracts of roots of the Sophora plant.

### Example 1

### MIC-3 In Vivo Experiment

Five groups of 5 male ICR derived mice weighing 22±2 grams are fasted overnight for 18 hours. Then, all of the mice undergo intragastrical inoculation of H. pylori in suspension at 3.0×10⁹ CFU/0.4 ml/mouse. A capsule containing test substance MIC-3, geranium oil and extracts of S. flavescenes, is dissolved in corn oil and adjusted into the final concentration of 30 mg/ml, 15 mg/ml and 5 mg/ml. MIC-3 (50, 150, and 300 mg/kg), vehicle control (corn oil 10 ml/kg) or positive control (omeprazole 1 mg/kg+clarithromycin 10 mg/kg) are administered orally to test animals one hour after the Helicobacter pylori inoculation, followed by dosing twice daily for 7 consecutive days. On the eighth day after infection, all animals with overnight fasting are sacrificed and the stomachs are dissected alone the creater curvature. Gastric ulceration is examined and scored at four levels with increasing degree of hemorrhage and severity of ulcerative lesions: 0=normal appearance, 1=mild red spots, 2=moderate red spots and/or hemorrhage spots, 3=marked hemorrhage spots. Reduction of concurrent vehicle control score values by 50 percent or more (≧50%) is considered significant. The result is shown in the table below.

Gastric Ulcer, Helicobacter pylori Bacteria, in Mice

| | | | | | Ulceration Score | | | | | | |
|---|---|---|---|---|---|---|---|---|---|---|---|
| | | | | | Individual | | | | | | |
| Treatment | Route | Dose | | N | 1 | 2 | 3 | 4 | 5 | Total | % Inhibition |
| Vehicle-Control | PO | 10 | ml/kg × 2 × 7 | 5 | 3 | 1 | 2 | 3 | 2 | 11 | — |
| (Corn Oil) | | | | | | | | | | | |
| PT#1028636 | PO | 50 | mg/kg × 2 × 7 | 5 | 3 | 2 | 1 | 1 | 0 | 7 | 36 |
| (MIC-3) | | | | | | | | | | | |
| (MIC-3 + TWEEN | PO | 150 | mg/kg × 2 × 7 | 5 | 0 | 0 | 1 | 1 | 3 | 5 | (55) |
| 80 ® + DDH₂O) | PO | 300 | mg/kg × 2 × 7 | 5 | 1 | 1 | 1 | 0 | 1 | 4 | (64) |
| Omeprazole + | PO | (1 + 10) | mg/kg × 7 | 5 | 2 | 0 | 0 | 1 | 1 | 4 | (64) |
| Clarithromycin | | | | | | | | | | | |

Significant reduction of gastric ulcer from H. pylori infection is achieved at the dosage of 150 mg/kg twice a day or 300 mg/kg/twice a day of MIC-3. There are various degrees of hemorrhage and severity of lesions on gastric mucosa in mice administered with vehicle control, MIC-3 at 50 mg/kg, 150 mg/kg, and 300 mg/kg, and positive control respectively.

### LD₅₀ Animal Experiment with Oral Administration

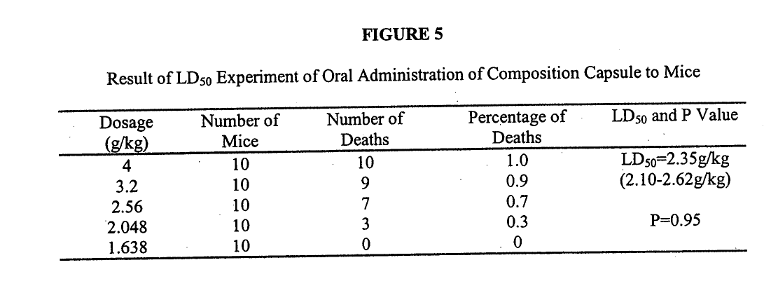

Fifty (50) ICR derived mice, half male and half female, weighing 18-22 grams, provided by animal laboratories of Anti-Bacterial Industrial Research Institute of Szechwan province, China, were used as test animals. The test solution was prepared by using 0.5% CMC to disintegrate the capsule, containing geranium oil and extracts from Sophora roots, and suspension solutions added to obtain the required concentration. The 50 mice were then divided into 5 groups, with 10 mice in each group (half male and half female). The 5 groups of mice were given the composition orally at various dosages of 4.000 g/kg, 3.200 g/kg, 2.560 g/kg, 2.048 g/kg, and 1.638 g/kg respectively. The dosages between the groups have a proportional value of 1:0.8. The drug was administered once to all the mice, and the mice were subsequently observed for 14 days for any death. On the third day after the drug administration, some mice start dying, and before death there was twitching, shortness of breath, and cessation of food intake. FIG. 5 shows the results of the experiment. According to the result, half of the tested mice will die at the dosage of 2.35 g/kg of composition capsule, and none of the tested mice died at the dosage of 1.638 g/kg, which is the maximum tolerated dosage for mice. The LD₅₀ dosage is 2.35 g/kg with a range of 2.10 to 2.62 g/kg (P=0.95).

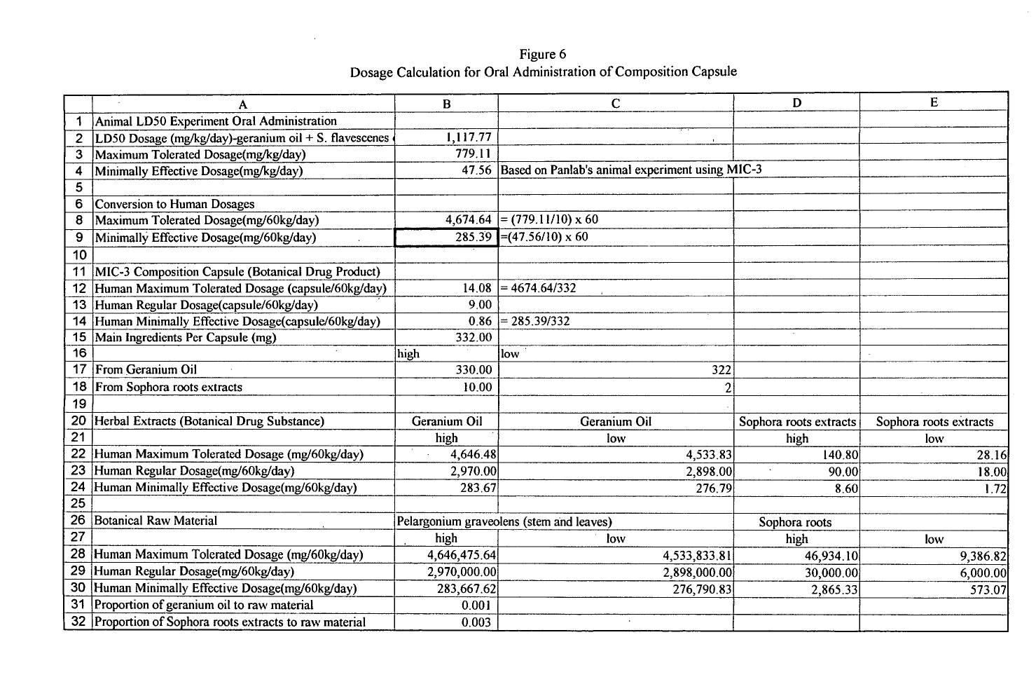

Result from LD₅₀ (50% lethality) experiment, in which the mice are administered orally with the composition, provides guidance on the range of safe dosages. The dosage of geranium oil and extracts from S. flavescenes alone, without the excipients, are calculated. A ratio is obtained, i.e. 698 g (weight of entire capsule) to Geranium oil (about 322 g) plus extracts from S. flavescenes (about 10 g) or 698 g to 332 g. The ratio of 332/698 multiplies the original LD50 dosage of entire capsule provides the dosage of geranium oil plus extracts from S. flavescenes only. See FIG. 6.

### Example 2

### MIC-1 In Vitro Experiment

Minimum inhibitory concentration is determined by the agar dilution method. 2 mg of test substance, composition of geranium oil and extracts from S. flavescenes prepared for injections (MIC-1), is dissolved and serially diluted in solvent (distilled water) to desired stock concentrations. For each concentration tested, a 10 μl aliquot is added to a 48-well plate containing 0.99 ml of Columbia agar base supplemented with 7% defibrinated rabbit blood. The inoculum of H. pylori (ATCC 43504) is prepared by suspending in brain heart infustion broth to a density of 5×10⁸ CFU/ml. A multiprong-incubating device is used to place approximately 5×10⁵ CFU/ml per spot onto the containing agent media. Thus, final maximal concentration of distilled water is 1% and the initial test substance concentration is 100 μg/ml. The plates are incubated at 37° C. for 72 hours in the microaerophilic condition (mixed gas N₂ 85%, CO₂ 10%, and O₂ 5%) and then visually examined and scored positive (+) for inhibition/eradication of colonies growth or negative (−) for no effect upon growth colonies. Vehicle-control, distilled water, and active reference agent, gentamicin, of 0.3 μg/ml are used as blank and positive controls, respectively. Each concentration is evaluated in duplicate.

The results of the experiment is set out in the table below:

| Assay Name | Route | n | Conc. | | Criteria | Result |
|---|---|---|---|---|---|---|
| Helicobacter pylori | in vitro | 2 | 10 | mg/ml | +/− | + |
| (MIC-1) | | 2 | 3 | mg/ml | +/− | + |
| | | 2 | 1000 | μg/ml | +/− | + |
| | | 2 | 300 | μg/ml | +/− | + |
| | | 2 | 100 | μg/ml | +/− | + |
| | | 2 | 30 | μg/ml | +/− | + |
| | | 2 | 10 | μg/ml | +/− | − |
| | | 2 | 3 | μg/ml | +/− | − |
| Helicobacter pylori | in vitro | 2 | 0.3 | μg/ml | +/− | + |
| (gentamicin) | | | | | | |

The concentration of 30 μg/ml or more achieves the result of inhibition/eradication of H. pylori growth.

### Example 3

### MIC-9 In Vitro Experiment

Minimum inhibitory concentration is determined by the agar dilution method. 2 mg of test substance, powder composition of geranium oil and extracts from S. flavescenes with a weight ratio of 30:1 of geranium oil to S. flavescenes (MIC-9), is dissolved and serially diluted in solvent (100% DMSO) to desired stock concentrations. For each concentration tested, a 10 μl aliquot is added to a 48-well plate containing 0.99 ml of Columbia agar base supplemented 7% defibrinated rabbit blood. The inoculum of H. pylori (ATCC 43504) is prepared by suspending in brain heart infustion broth to a density of 5×10⁸ CFU/ml. A multiprong-incubating device is used to place approximately 5×10⁵ CFU/ml per spot onto the containing agent media. Thus, final maximal concentration of DMSO is 1% and the initial test substance concentration is 100 μg/ml. The plates are incubated at 37° C. for 72 hours in the microaerophilic condition (mixed gas N₂ 85%, CO₂ 10%, and O₂ 5%) and then visually examined and scored positive (+) for inhibition/eradication of colonies growth or negative (−) for no effect upon growth colonies. Vehicle-control, 100% DMSO, and active reference agent, gentamicin, of 0.3 μg/ml are used as blank and positive controls, respectively. Each concentration is evaluated in duplicate.

The result of the experiment is set out in the table below:

| Assay Name | Route | n | Conc. | | Criteria | Result |
|---|---|---|---|---|---|---|
| Helicobacter pylori | in vitro | 2 | 30 | mg/ml | +/− | + |
| (MIC-9) | | 2 | 10 | mg/ml | +/− | + |
| | | 2 | 3 | mg/ml | +/− | + |
| | | 2 | 1000 | μg/ml | +/− | + |
| | | 2 | 300 | μg/ml | +/− | + |
| | | 2 | 100 | μg/ml | +/− | − |
| | | 2 | 30 | μg/ml | +/− | − |
| | | 2 | 10 | μg/ml | +/− | − |
| Helicobacter pylori | in vitro | 2 | 0.3 | μg/ml | +/− | + |
| (Gentamicin) | | | | | | |

The concentration of 300 μg/ml or more achieves the result of inhibition/eradication of H. pylori growth.

### Example 4

### MIC-10 In Vitro Experiment

Minimum inhibitory concentration is determined by the agar dilution method. 2 mg of test substance, powder composition of geranium oil and extracts from S. tonkinensis with a weight ratio of 30:1 of geranium oil to S. tonkinensis (MIC-10), is dissolved and serially diluted in solvent (100% DMSO) to desired stock concentrations. For each concentration tested, a 10 μl aliquot is added to a 48-well plate containing 0.99 ml of Columbia agar base supplemented 7% defibrinated rabbit blood. The inoculum of H. pylori (ATCC 43504) is prepared by suspending in brain heart infustion broth to a density of 5×10⁸ CFU/ml. A multiprong-incubating device is used to place approximately 5×10⁵ CFU/ml per spot onto the containing agent media. Thus, final maximal concentration of DMSO is 1% and the initial test substance concentration is 100 μg/ml. The plates are incubated at 37° C. for 72 hours in the microaerophilic condition (mixed gas N₂ 85%, CO₂ 10%, and O₂ 5%) and then visually examined and scored positive (+) for inhibition/eradication of colonies growth or negative (−) for no effect upon growth colonies. Vehicle-control, 100% DMSO, and active reference agent, gentamicin, of 0.3 μg/ml are used as blank and positive controls, respectively. Each concentration is evaluated in duplicate.

The result of the experiment is set out in the table below:

| Assay Name | Route | n | Conc. | | Criteria | Result |
|---|---|---|---|---|---|---|
| Helicobacter pylori | in vitro | 2 | 30 | mg/ml | +/− | + |
| (MIC-10) | | 2 | 10 | mg/ml | +/− | + |
| | | 2 | 3 | mg/ml | +/− | + |
| | | 2 | 1000 | μg/ml | +/− | + |
| | | 2 | 300 | μg/ml | +/− | + |
| | | 2 | 100 | μg/ml | +/− | − |
| | | 2 | 30 | μg/ml | +/− | − |
| | | 2 | 10 | μg/ml | +/− | − |
| Helicobacter pylori | in vitro | 2 | 0.3 | μg/ml | +/− | + |
| (Gentamicin) | | | | | | |

The concentration of 300 μg/ml or more achieves the result of inhibition/eradication of H. pylori growth.

### Example 5

### MIC-11 In Vitro Experiment

Minimum inhibitory concentration is determined by the agar dilution method. 2 mg of test substance, powder mixture with 10% of S. tonkinensis powders and 90% geranium oil powders (MIC-11), is dissolved and serially diluted in solvent (100% DMSO) to desired stock concentrations. For each concentration tested, a 10 μl aliquot is added to a 48-well plate containing 0.99 ml of Columbia agar base supplemented 7% defibrinated rabbit blood. The inoculum of H. pylori (ATCC 43504) is prepared by suspending in brain heart infustion broth to a density of 5×10⁸ CFU/ml. A multiprong-incubating device is used to place approximately 5×10⁵CFU/ml per spot onto the containing agent media. Thus, final maximal concentration of DMSO is 1% and the initial test substance concentration is 100 μg/ml. The plates are incubated at 37° C. for 72 hours in the microaerophilic condition (mixed gas N₂ 85%, CO₂ 10%, and O₂ 5%) and then visually examined and scored positive (+) for inhibition/eradication of colonies growth or negative (−) for no effect upon growth colonies. Vehicle-control, 100% DMSO, and active reference agent, Gentamicin, of 0.3 μg/ml are used as blank and positive controls, respectively. Each concentration is evaluated in duplicate.

The result of the experiment is set out in the table below:

| Assay Name | Route | n | Conc. | | Criteria | Result |
|---|---|---|---|---|---|---|
| Helicobacter pylori | in vitro | 2 | 30 | mg/ml | +/− | + |
| (MIC-11) | | 2 | 10 | mg/ml | +/− | + |
| | | 2 | 3 | mg/ml | +/− | + |
| | | 2 | 1000 | μg/ml | +/− | + |
| | | 2 | 300 | μg/ml | +/− | + |
| | | 2 | 100 | μg/ml | +/− | − |
| | | 2 | 30 | μg/ml | +/− | − |
| | | 2 | 10 | μg/ml | +/− | − |
| Helicobacter pylori | in vitro | 2 | 0.3 | μg/ml | +/− | + |
| (Gentamicin) | | | | | | |

The concentration of 300 g/ml or more achieves the result of inhibition/eradication of H. pylori growth.

### Example 6

### MIC-12 In Vitro Experiment

Minimum inhibitory concentration is determined by the agar dilution method. 2 mg of test substance, powder mixture with 30% of S. tonkinensis powders and 70% geranium oil powders (MIC-12), is dissolved and serially diluted in solvent (100% DMSO) to desired stock concentrations. For each concentration tested, a 10 μl aliquot is added to a 48-well plate containing 0.99 ml of Columbia agar base supplemented 7% defibrinated rabbit blood. The inoculum of H. pylori (ATCC 43504) is prepared by suspending in brain heart infustion broth to a density of 5×10⁸ CFU/ml. A multiprong-incubating device is used to place approximately 5×10⁵ CFU/ml per spot onto the containing agent media. Thus, final maximal concentration of DMSO is 1% and the initial test substance concentration is 100 μg/ml. The plates are incubated at 37° C. for 72 hours in the microaerophilic condition (mixed gas N₂ 85%, CO₂ 10%, and O₂ 5%) and then visually examined and scored positive (+) for inhibition/eradication of colonies growth or negative (−) for no effect upon growth colonies. Vehicle-control, 100% DMSO, and active reference agent, gentamicin, of 0.3 μg/ml are used as blank and positive controls, respectively. Each concentration is evaluated in duplicate.

The result of the experiment is set out in the table below:

| Assay Name | Route | n | Conc. | | Criteria | Result |
|---|---|---|---|---|---|---|
| Helicobacter pylori | in vitro | 2 | 30 | mg/ml | +/− | + |
| (MIC-12) | | 2 | 10 | mg/ml | +/− | + |
| | | 2 | 3 | mg/ml | +/− | + |
| | | 2 | 1000 | μg/ml | +/− | + |
| | | 2 | 300 | μg/ml | +/− | + |
| | | 2 | 100 | μg/ml | +/− | − |
| | | 2 | 30 | μg/ml | +/− | − |
| | | 2 | 10 | μg/ml | +/− | − |
| Helicobacter pylori | in vitro | 2 | 0.3 | μg/ml | +/− | + |
| (Gentamicin) | | | | | | |

The concentration of 3001 g/ml or more achieves the result of inhibition/eradication of H. pylori growth.

H. pylori growth refers both to colonies or CFU of H. pylori and H. pylori cell(s) found in vivo. Several methods may be used to diagnose H. pylori growth in vivo from H. Pylori infection. Serological tests that measure specific H. pylori IgG antibodies can determine if a person has been infected. The sensitivity and specificity of these assays range from 80% to 95% depending upon the assay used. Another diagnostic method is the breath test, wherein the patient is given either ¹³C- or ¹⁴C-labeled urea to drink. H. pylori metabolizes the urea rapidly, and the labeled carbon is absorbed. This labeled carbon can then be measured as CO₂ in the patient's expired breath to determine whether H. pylori is present. The sensitivity and specificity of the breath test ranges from 94% to 98%. Upper esophagogastroduodenal endoscopy may also be employed. During endoscopy, biopsy specimens of the stomach and duodenum are obtained and the diagnosis of H. pylori can be made by several methods. One method is the biopsy urease test, a colorimetric test based on the ability of H. pylori to produce urease. Also, the organism may be identified histologically. Finally, biopsy specimens can be cultured for H. pylori. Upon locating the H. pylori growth in vivo with an assay, the composition of the present invention may be delivered to the H. pylori growth via administration to the host.

### Example 7

### MIC-9 In Vitro Experiment

Na⁺/K⁺-ATPase is obtained from dog kidney. Test compound, powder composition of geranium oil and extracts from S. flavescenes with a weight ratio of 30:1 of geranium oil to S. flavescenes (MIC-9), is preincubated with 80 mM Tris-HCl buffer pH 7.4 containing 160 mM NaCl, 25 mM KCl, 5.3 mM MgCl₂, 1.3 mM EDTA and enzyme (0.02 units) for 20 minutes at 37° C. The reaction is initiated by addition of ATP (2 mM final) and further incubated for 15 minutes after which the reaction is stopped by addition of 2.5 N HClO₄. The reaction product of inorganic phosphate is determined by spectrophotometer with the addition of Fiske-Subbarow reagent and the reading at 660 nm. Compounds are screened at 10 [symbol] M. Each concentration is evaluated in duplicate.

| Assay Name | n | Conc. | | % Inhibition | IC₅₀ |
|---|---|---|---|---|---|
| ATPase, Na⁺/K⁺ | 2 | 3 | mg/ml | 113 | 302 μg/ml |
| (MIC-9) | 2 | 300 | μg/ml | 58 | |
| | 2 | 30 | μg/ml | 9 | |
| | 2 | 3 | μg/ml | 8 | |
| | 2 | 0.3 | μg/ml | 2 | |

The concentration of 302 μg/ml or more can inhibit the enzymatic activities of half of the of Na⁺/K⁺-ATPase.

### Example 8

### MIC-10 In Vitro Experiment

Na⁺/K⁺-ATPase is obtained from dog kidney. Test compound, powder composition of geranium oil and extracts from S. tonkinensis with a weight ratio of 30:1 of geranium oil to S. tonkinensis (MIC-10), is preincubated with 80 mM Tris-HCl buffer pH 7.4 containing 160 mM NaCl, 25 mM KCl, 5.3 mM MgCl₂, 1.3 mM EDTA and enzyme (0.02 units) for 20 minutes at 37° C. The reaction is initiated by addition of ATP (2 mM final) and further incubated for 15 minutes after which the reaction is stopped by addition of 2.5 N HClO₄. The reaction product of inorganic phosphate is determined by spectrophotometer with the addition of Fiske-Subbarow reagent and the reading at 660 mm. Compounds are screened at 10 [symbol] M. Each concentration is evaluated in duplicate.

| Assay Name | n | Conc. | | % Inhibition | IC₅₀ |
|---|---|---|---|---|---|
| ATPase, Na⁺/K⁺ | 2 | 3 | mg/ml | 113 | 360 μg/ml |
| (MIC-10) | 2 | 300 | μg/ml | 46 | |
| | 2 | 30 | μg/ml | 3 | |
| | 2 | 3 | μg/ml | 4 | |
| | 2 | 0.3 | μg/ml | 7 | |

The concentration of 360 μg/ml or more can inhibit the enzymatic activities of half of the of Na⁺/K⁺-ATPase.

### Example 9

### MIC-11 In Vitro Experiment

Na⁺/K⁺-ATPase is obtained from dog kidney. Test compound, powder mixture with 10% of S. tonkinensis powders and 90% geranium oil powders (MIC-11), is preincubated with 80 mM Tris-HCl buffer pH 7.4 containing 160 mM NaCl, 25 mM KCl, 5.3 mM MgCl₂, 1.3 mM EDTA and enzyme (0.02 units) for 20 minutes at 37° C. The reaction is initiated by addition of ATP (2 mM final) and further incubated for 15 minutes after which the reaction is stopped by addition of 2.5 NHClO₄. The reaction product of inorganic phosphate is determined by spectrophotometer with the addition of Fiske-Subarrow reagent and reading at 660 nm. Compounds are screened at 10 [symbol] M. Each concentration is evaluated in duplicate.

| Assay Name | n | Conc. | | % Inhibition | IC₅₀ |
|---|---|---|---|---|---|
| ATPase, Na⁺/K⁺ | 2 | 3 | mg/ml | 90 | 130 μg/ml |
| (MIC-11) | 2 | 300 | μg/ml | 73 | |
| | 2 | 30 | μg/ml | 15 | |
| | 2 | 3 | μg/ml | 10 | |
| | 2 | 0.3 | μg/ml | 2 | |

The concentration of 130 μg/ml or more can inhibit the enzymatic activities of half of the of Na⁺/K⁺-ATPase.

### Example 10

### MIC-12 In Vitro Experiment

Na⁺K⁺-ATPase is obtained from dog kidney. Test compound, powder mixture with 30% of S. tonkinensis powders and 70% geranium oil powders (MIC-12), is preincubated with 80 mM Tris-HCl buffer pH 7.4 containing 160 mM NaCl, 25 mM KCl, 5.3 mM MgCl₂, 1.3 mM EDTA and enzyme (0.02 units) for 20 minutes at 37° C. The reaction is initiated by addition of ATP (2 mM final) and further incubated for 15 minutes after which the reaction is stopped by addition of 2.5 N HClO₄. The reaction product of inorganic phosphate is determined by spectrophotometer with the addition of Fiske-Subbarow reagent and the reading at 660 nm. Compounds are screened at 10 μM. Each concentration is evaluated in duplicate.

| Assay Name | n | Conc. | | % Inhibition | IC₅₀ |
|---|---|---|---|---|---|
| ATPase, Na⁺/K⁺ | 2 | 3 | mg/ml | 103 | 234 μg/ml |
| (MIC-12) | 2 | 300 | μg/ml | 62 | |
| | 2 | 30 | μg/ml | −1 | |
| | 2 | 3 | μg/ml | 3 | |
| | 2 | 0.3 | μg/ml | 1 | |

The concentration of 234 [symbol] g/ml or more can inhibit the enzymatic activities of half of the Na⁺/K⁺-ATPase.

### Example 11

### MC-20 In Vivo Experiment

Seven groups of 5 male ICR derived mice weighing 24±2 grams were fasted for 18 hours before the intragastric inoculation of Helicobacter pylori (clinical isolate strain) in suspension at 1.5×10⁹ CFU/0.4 ml/mouse.

MIC-17 (50 mg/kg and 100 mg/kg of geranium oil) and MIC-20 (24.5 mg/kg of citronellol) were dissolved in 2% TWEEN 80® in 0.9% NaCl solution as working solution which was adjusted to final concentration of 898 and 449 mg/ml for 50 mg/kg and 100 mg/kg of MIC-17 respectively and 858 mg/ml for MIC-20.

MIC-17 at dose of 100 and 50 mg/kg, MIC-20 at dose of 24.5 mg/kg, MIC-18 in liquid form at 61.77 μl/kg (equivalent to 50 mg/kg of geranium oil plus extracts of S. tonkinensis containing 0.058 mg/kg of matrine dosage), MIC-19 in liquid form at 116.58 μl/kg (equivalent to 50 mg/kg geranium oil plus extracts of S. tonkinensis containing 0.58 mg/kg of matrine dosage), and the vehicle (2% TWEEN 80® in 0.9% NaCl solution), as a negative control, at 10 ml/kg were administered orally to test animals one hour after the Helicobacter pylori inoculation, followed by a second dosing at 7 hours later. Subsequently, test substances and vehicle were each administered orally twice daily (9:00 A.M. and 16:00 P.M.) for 6 consecutive days. omeprazole 1 mg/kg and clarithromycin 10 mg/kg in combination was used as a positive control agent and was administered orally to test animals once daily (9:00 A.M.) for 7 consecutive days.

Seven days after infection, on day eight, all animals with overnight fasting were sacrificed and the stomachs were dissected along the greater curvature. Gastric ulceration was scored at four levels according to the degree of hemorrhage and the severity of ulcerative lesions: 0=normal appearance, 1=mild red spots, 2=moderate red spots and/or hemorrhage spots, 3=marked hemorrhage spots. Reduction of concurrent vehicle control score values by 50 percent or more (≧50%) is considered significant.

MIC-17 at 100 mg/kg, MIC-18 at 61.77 μl/kg, MIC-19 at 116.58 μl/kg and MIC-20 at 24.5 mg/kg, respectively, caused a significant decrease (≧50%) in gastric ulceration relative to the vehicle control value. MIC-17 at 50 mg/kg was associated with a moderate (36%) but non-significant reduction in ulcers in comparison with the vehicle control group. The positive control of omeprazole (1 mg/kg) in combination with clarithromycin (10 mg/kg) caused a significant decrease (73%) in ulceration score relative to the vehicle-treated group.

| | | | | | Ulceration Score | | | | | | |
|---|---|---|---|---|---|---|---|---|---|---|---|
| | | | | | Individual | | | | | | |
| Treatment | Route | Dose | | N | 1 | 2 | 3 | 4 | 5 | Total | % Inhibition |
| Vehicle-Control | PO | 10 | ml/kg × 2 × 7 | 5 | 3 | 3 | 2 | 1 | 2 | 11 | — |
| (2% TWEEN 80 ®/0.9% | | | | | | | | | | | |
| NaCl) | | | | | | | | | | | |
| PT# 1042220 (MIC-17) | PO | 100 | ml/kg × 2 × 7 | 5 | 1 | 0 | 0 | 2 | 0 | 3 | (73) |
| (MIC-13) | PO | 50 | ml/kg × 2 × 7 | 5 | 3 | 2 | 1 | 1 | 0 | 7 | 36 |
| PT# 1042221 (MIC-18) | PO | 61.77 | \hat{ }l/kg × 2 × 7 | 5 | 1 | 2 | 0 | 1 | 0 | 4 | (64) |
| PT# 1042222 (MIC-19) | PO | 116.58 | \hat{ }l/kg × 2 × 7 | 5 | 0 | 1 | 1 | 1 | 0 | 3 | (73) |
| PT# 1042223 (MIC-20) | PO | 24.5 | ml/kg × 2 × 7 | 5 | 0 | 2 | 1 | 0 | 0 | 3 | (73) |
| Omeprazole + | PO | (1 + 10) | ml/kg × × 7 | 5 | 1 | 0 | 0 | 1 | 1 | 3 | (73) |
| Clarithromycin+ | | | | | | | | | | | |

Test substances and vehicle control (2% TWEEN 80® in 0.9% NaCl solution) were each administered orally to test animals twice daily for 7 consecutive days. The Helicobacter pylori (1.5×109 CFU/0.4 ml/mouse) inoculation was applied one hour before the first dosing on day 1. All overnight-fasted animals were sacrificed on day 8 (7 days after infection) and the stomachs were dissected along greater curvature. Reduction of concurrent vehicle control score values by 50 percent or more (≧50%) is considered significant.

Citronellol alone, MIC-20, can have the same strong ulceration inhibition effect as MIC-17, MIC-19, and the positive control—omeprazole plus clarithromycin.

1-63. (canceled)

64. A method of treating a mammal infected with H. pylori comprising administering to the mammal a composition comprising citronellol.

65. The method of claim 64, wherein the mammal is a human.

66. The method of claim 64, wherein the composition is administered orally.

67. The method of claim 64, wherein the composition is in a dosage about 25 mg/kg.

68. The method of claim 65, wherein the composition is in a dosage about 150 mg/60 kg.

69. The method of claim 67 or 68, wherein the dosage is administered twice a day.

70. A method of inhibiting H. pylori growth comprising delivering a composition to H. pylori comprising citronellol.

71-75. (canceled)
